(19) United States
(12) Patent Application Publication
(10) Pub. No.: US 2005/0020482 A1
(43) Pub. Date: Jan. 27, 2005

(54) Methods for modulating gap junctions

(76) Inventors: Jennifer Phipps, Chelsea (CA); Loubaba Belbaraka, Ottawa (CA); Herve Jouishomme, Venissieux (FR); Saba Siddiqi, Ottawa (CA); Jaafar Belgoudi, Ottawa (CA); Jennifer Arnold, Ottawa (CA); Suzanne Lacelle, Orleans (CA)

Correspondence Address: NATIONAL RESEARCH COUNCIL OF CANADA;1500 MONTREAL ROAD, BLDG M-58, ROOM EG12, OTTAWA, ONTARIO, K1A 0R6, CA

(21) Appl. No.: 10/480,685

(22) PCT Filed: Jun. 14, 2002
(86) PCT No.: PCT/CA02/00879

(30) Foreign Application Priority Data
Jun. 15, 2001 (US) 60/298102

(51) Int. Cl. A61K 38/00; A61K 31/70

(52) U.S. Cl. 514/2; 514/23

(57) ABSTRACT

A method for modulating gap junctions is provided. In a first step, the transcription of connexin is enhanced and the gap junction is relocalized at the cell membrane; in a second step, gap junction function is restored by properly gating the channel and/or inducing apoptosis. The method preferably comprises a providing a cell or an animal with a first compound for increasing the activity of protein kinase A (PKA) and in the second step providing the cell or animal with a second compound for specifically inhibiting or activating one or more protein kinase C (PKC) isoforms and/or for specifically inhibiting p38 MAP kinase.

### FIELD OF THE INVENTION

This invention relates to methods for modulating gap junctions.

### BACKGROUND OF THE INVENTION

It has been established that in many chronic diseases, such as cancer, cardiac conditions, and central nervous system diseases, gap junction mediated intercellular communications are dysfunctional (see for example Bruzzone, R. et al, European Journal of Neuroscience, 9:1-6 (1997); Holder, J. et al., Cancer Research, 53:3475-3485 (1993); Yamasaki, H., Environmental health perspectives, 93:191-197 (1991), Peters, N., Clinical Science, 90:447-452 (1996); Mesnil, M. et al., M/S Mini-Synthése, 12:1435-1438 (1996); Dilber, M. et al., Gene Therapy, 4:273-274 (1997); Pal, J. et al., American Physiological Society, 1443-1446 (1999); Marques Jr., W. et al., Journal Of Neurology, Neurosurgery And Psychiatry, 66:803-804 (1999); Heller, S. et al., Nature Medicine, 4:560-561 (1998); and Holt, J. et al, Neuron, 22:217-219 (1999)).

A gap junction is a membrane structure detectable at points of contact between adjacent cells. Gap junctions serve as passageways or channels between the interiors of contiguous cells, and mediate intercellular communication by the passage of small molecules from the cytoplasm of one cell to that of adjacent cells.

Gap junctions are composed of clusters of membrane proteins collectively termed connexins which form structures called connexons. The proteins are peripherally disposed around a central channel. Gap junction transmembrane passages are formed when a connexon of one cell aligns with a connexon of an adjacent cell. In this way, transmembrane intercellular pathways are formed that permit the passage of molecules between coupled cells.

The diameter of the connexon channels is about 1.5 to 2 nm. This diameter allows only small molecule substances of less than approximately 1,000 daltons, such as ions, sugars, nucleic acids, amino acids, fatty acids, and small peptides, to pass but not large molecules, such as proteins, complex lipids, polysaccharides and polynucleotides.

The protein subunits of connexons may vary from cell to cell. The connexins (Cx), form a multi-gene family whose members are distinguished according to their predicted molecular mass in kDa (e.g. Cx32, Cx43). Connexins are expressed in a cell-, tissue-, and developmentally-specific manner. See Beyer et al., J. Membr. Biol., 116:187-194 (1990); Dermietzel, R. et al., Anat. Embryol., 182:517-258 (1990; Warner, A., Seminars in Cell Biology, 3:81-91 (1992); Kumar, N. M. et al., Seminars in Cell Biology, 3:3-16 (1992). For instance, Cx43 is the predominant connexin expressed in cardiac muscle and in liver epithelial cells. In adult liver parenchymal cells, the predominant connexins are Cx32 and Cx26; non parenchymal liver cells express other connexins. Each connexin forms channels with different conductance, regulatory, and permeability properties. In those tissues where more than one connexin is expressed, gap junctions may contain more than one connexin. However, it is not known whether individual connexons may be comprised of more than one connexin type.

Gap junctions do not necessarily remain open. Elevation of the intracellular level of calcium ions, among other factors, leads to a graded closure of gap junctions. In healthy, normal cells, these channels are fully open when the calcium ion level is less than 10⁻⁷ M and are shut when the level of this ion is higher than 5×10⁻⁵ M. As the concentration of calcium ion increases in this range, the effective diameter of gap junctions decreases so that they become impermeable first to larger molecules. When the normally very low (10⁻⁸M) intracellular calcium levels rise, the gap junction proteins undergo conformational changes that close the gap. In certain diseases, the gap junction gating is dysfunctional resulting in numerous problems.

Many solid tumors, for instance, have deficient gap junctions, a condition that prevents the communication among cancer cells and between the cancer cells and healthy surrounding cells leading to an alteration in the intracellular levels of calcium, and other small molecules. This leads to an upset in normal tissue homeostasis and favours uncontrolled cell proliferation.

One method of combating tumours is proposed in U.S. Pat. No. 6,149,904 to Fick et al. issued on Nov. 21, 2000. Fick et al. seek to provide cells that are genetically engineered to increase their ability to interact with and to inhibit tumour cells. Described is a method of providing engineered cells that express a heterologous nucleic acid that encodes a connexin and that contains a pro-drug activating gene. The target tumour cells are contacted with the engineered cells to form functional gap junctions between the two groups of cells. In this way, the invention aims to have the therapeutic molecule pass through a gap junction into the target tumour cell. However, this method seeks to employ gene therapy associated with a toxin to stop proliferation of the tumour rather than re-establishing a more normal gap junction function, as a method of controlling the tumour.

The ability of adjacent cells to form gap junctions may depend on a number of factors including the ability of cells to interact with their neighbours (reportedly requiring the presence of compatible cell adhesion molecules), the level of connexin expressed, whether the connexins expressed by one cell are capable of forming a connexon that is capable of linking with a connexon of a second cell to form a functional channel, and whether a molecule, such as a carcinogen or the product of an oncogene is expressed in a cell and interferes with the normal signaling pathways. Most oncogenes or carcinogens alter the gating of the gap junction channel by affecting the activity of protein kinases that use connexins as a substrate (such as, PKA, PKCs, c-src, MAPK).

By controlling gap junctions, communication between cells in diseased tissue can be restored. As a consequence, the tissue or organ may regain normal function and regain growth homeostasis. For instance, in the case of tumours, re-establishment of intercellular communication and/or increase in cell coupling between malignant cells and the surrounding healthy cells restores the normal phenotype of malignant cells and leads to growth arrest.

### SUMMARY OF THE INVENTION

It is, thus desirable to provide a method for modulating gap junction channels in order to treat chronic diseases and disorders characterized by dysfunctional gap junctions, or increase “bystander effects”. Examples are cancer, such as neuroblastoma and other malignant solid tumours, cardiac conditions, such as arrhythmia, and central nervous system diseases.

In a first aspect, the invention provides a method step of modulating gap junction mediated intercellular communication in mammalian cells characterized by dysfunctional gap junction communication, comprising providing the cells with a compound that re-localizes the gap junctions at the cells' membrane. In one embodiment, this is effected, by re-locating connexins in the cells' cytoplasm from a perinuclear location, to the cell membrane. In another embodiment, the expression of connexin is also increased. In yet another embodiment, the compound is an activator of a selected protein kinase A(PKA) isoform.

In a second aspect, the invention provides a method step of modulating gap junction mediated intercellular communication in mammaalian cells characterized by dysfunctional gap junction communication, comprising providing the cells with a compound that restores gap junction gating. In one embodiment, the compound is a modulator for specifically inhibiting or activating one or more selected PKC isoforms. The two steps may be performed, individually, sequentially or simultaneously, depending upon the requirement. In some cases, only one step is required, while in others, both steps may be required. It may be enough to increase the level of expression of connexin, and/or localize the connexin to the cells' membrane e.g. by activation of a selected PKA isoform. In other cases, the gap junctions may be properly localized at the cells' membrane, but not properly gated to permit effective intercellular communication. In one situation, the gating with surrounding healthy tissue cannot be made. In another situation, the gating is made with surrounding tissue with which it is not supposed to communicate. In such cases, only the second type of drug candidate e.g. a selected modulator on one or more PKC isoforms, would be required.

In another aspect, the invention provides a method of modulating gap junction operation in a cell or in an animal comprising the steps of providing the cell or animal with a first compound for increasing the activity of PKA and providing the cell or animal with a second compound for inducing apoptosis. Regarding apoptosis, we rely upon our observations of cytoplasmic leakage.

A method of modulating gap junction operation in a cell, comprising the steps of:
(a) providing the cell with a protein kinase A (PKA) activator; and
(b) providing the cell with a p38 MAP kinase inhibitor.

According to yet another aspect of the invention, an assay for testing drug candidates, for treating diseases, disorders or conditions characterized by dysfunctional gap junction mediated intercellular communication, and/or which exhibit modulation of the activity of protein kinase A and/or protein kinase C is also provided. In one embodiment, the method comprises, a step of administering a drug candidate compound to cells having dysfunctional gap junction mediated intercellular communication and /or abnormal localization within the cells of connexin, and/or a low level of connexin expression. An estimation of the increase of connexin transcription by quantitative Western blot and a re-localization of the connexin to the cells's membrane, as determined e.g. by immunostaining, followed by microscope imaging analysis, denotes a positive result.

In another embodiment, the method comprises, a step of administering a drug candidate compound to cells having dysfunctional gap junction mediated intercellular communication. An observation, of an improvement in gap junction mediated intercellular communication as measured e.g. by scrape loading dye transfer assay, and/or a measurement of inhibition or activation of one or more selected PKC isoforms, denotes a positive result.

In yet another aspect, a method of treating a cancer tumour in a patient is provided, comprising the steps of:
(a) providing the cell with a protein kinase A (PKA) activator; and
(b) providing the cell with modulator for specifically inhibiting or activating one or more PKC isoforms.

The control of gap junctions in many types of tumours, such as neuroblastoma, results in the down-regulation of the tumour growth and the restoration of the normal cellular phenotype.

Other features and advantages of the invention will become apparent from the following description and from the claims.

### BRIEF DESCRIPTION OF THE DRAWINGS

The invention will now be explained by way of example only and with reference to the attached drawing in which:

FIG. 1 shows a confocal microscope image of abnormal perinuclear and cytoplasmic localization of Cx43 in IMR32 cells, a defect common to some other malignancies;

FIG. 2 shows electrophoresis and results from immunological detection demonstrating a Cx43 increase in expression following 8-bromo-cAMP (a PKA activator) treatment of IMR32 cells.

FIGS. 3A to 3F show an immunostain of SHSY5Y neuroblastoma cells after treatment with 8-bromo-cAMP in which the normal localization of Cx43 at the cell border is regained.

FIGS. 4A to 4D show in graphical form the control of growth of cell populations of IMR32 analyzed by FACS upon 8-bromo-cAMP supplementation;

FIGS. 5A to 5D and 6A to 6D show experiments using scrape loading dye transfer assay to estimate gap junction function; 1) Control untreated cells are gap junction deficient. 2) 8-Br-cAMP treatment increases dye transfer by a factor to 2 to 4 times 3) the full restoration of gap junction function following further specific inhibition of PKC alpha and beta 1,+beta 2.; the PKC isoform activity was inhibited selectively by introduction of selective Mab into the cells. Two different types of images of neuroblastoma cells after various treatments.

FIGS. 7A to 7D and 8A to 8D show the effect of the introduction inside the cells of specific anti betal, beta 2 and alpha+beta isoforms and the level of restoration of gap junction function as assessed by scrape loading dye transfer assay two different types of images of neuroblastoma cells. after various treatments. It confirms the positive regulatory effect of PKC alpha and beta 1, 2 to a lesser extent, on gap junction channel gating.

FIGS. 9A and 9B show the restoration of gap junction channel activity on IMR32 cells treated or not with an inhibitor of the p38 MAP kinase. The channels function was assessed by a scrape loading assay.

FIG. 10 shows in graphical form the effect of PCK alpha antisense on the growth of IMR32 cells. It confirms that growth limitation is associated with restoration of gap junction function (refer FIGS. 5 to 8).

### DETAILED DESCRIPTION OF THE INVENTION

In neuroblastoma cells, as it is the case in other cancers and certain other diseases, the Applicant has shown that abnormal gap junction function is due to abnormal localization of the gap junctions around the nucleus rather that on the cell plasma membrane, as well as improper gating of the gap junctions. In order to provide proper gap junction function in such diseases, it is beneficial to increase the expression of connexins, to properly localize the connexin and hence the gap junctions at the cell membrane, and to properly gate the gap junctions. By properly gating the gap junctions, we mean to restore proper function and capability for intercellular communication and growth limitation.

Thus, in one aspect the invention provides a two-step method. In a first step, the transcription of connexin is enhanced and the gap junction is relocalized at the cell membrane; in a second step, gap junction function is restored by properly gating the channel. This also drastically limits the cells proliferation, and restores normalcy. The method preferably comprises providing a cell or an animal or a human with a first compound for increasing the activity of a selected isoform of protein kinase A (PKA), and in the second step providing the cell or animal with a second compound for specifically inhibiting or activating one or more selected protein kinase C (PKC) isoforms. In another aspect, the invention provides a method comprising the specific inhibition of the p38 MAP kinase treatment of neuroblastoma cells. Such treatment fully restores gap junction channels function and induces apoptosis.

Protein kinases are a group of enzymes which modulate the activities of a variety of proteins in different cells by phosphorylating them. Protein kinase A (PKA) is a cAMP-dependent kinase that initiates the transfer from ATP of a phosphate ion onto either a serine or threonine group on the target protein. PKA activity regulates many cellular processes including cell growth, cell differentiation, ion-channel conductivity, gene transcription, synaptic release of neurotransmitters, and memory. The PKA family includes two primary isoforms ie PKA sub-type I and PKA sub-type II.

Protein kinase C (PKC) is a key signaling enzyme. It transduces cellular signals following activation by its effectors derived from lipid hydrolysis. This 80 kDa enzyme is recruited to the plasma membrane upon activation. The enzyme is activated by diacylglycerol, calcium and phospholipids and is thought to undergo a conformational change upon binding to the membrane. PKC phosphorylates a variety of target proteins that activate cascades of phosphorylating enzymes. They control growth and cellular ditterentiation as well as numeral otner Key ceiiuar processes. 11 isoforms form the PKC family, and these isoforms serve different functions in the cells.

Exemplary of cancer tumours, the Applicant has studied neuroblastoma cells. The Applicant has shown that gap junction function is deficient in neuroblastoma cells originating from different tumor types, and has also shown that gap junction channels in neuroblastoma are formed by Cx43.

To accomplish the first step, PKA is activated, such as by activating an isoform of PKA, for example an isoform of PKA subtype I or II. Activation of PKA enhances the transcription of intracellular Cx43. The Applicant has further shown that activation of PKA also induces the transfer of Cx43 from its abnormal location around the nucleus to the normal location on the cell membrane. Other PKA isoforms may also enhance the transcription and transfer of connexins in other systems. Activation of PKA is known in the art. It can be accomplished with a variety of compounds such as 8-bromo-cAMP and forskolin; the latter can be used in vivo.

However, even when located at the membrane, intercellular communication may remain limited. For instance, many of the gap junctions may remain closed. Thus, the method further involves restoring gap junction gating/function. Gap junction function may be restored in several ways, in accordance with the invention.

In one embodiment, specific inhibition of one or more PKC isoforms can be used. The Applicant has identified three isoforms of PKC, namely the alpha and beta 1, 2 isoforms, the inhibition of which, in neuroblastoma, contributes to open up gap junctions. The opening of the gap junctions is associated with growth restriction and the induction of differentiation of neuroblastoma cells. Alternatively, the activation of PKC could be important in certain systems for restoring gap junction function (as in colon cancer, for example).

The method further encompasses modulating different isoforms of PKC, as desired. Different connexins are involved in each tissue, and cause different diseases when defective. As each of these connexins may be modulated by different isoforms of PKC, different isoforms of PKC are anticipated to be important depending on the specific disease or tissue.

As mentioned above, PKC is responsible for numerous functions, specific tasks being performed by selective isoforms. Thus, an important aspect of this invention is that the activation or inhibition of PKC be done specifically targeting only the selected PKC isoforms. Numerous methods have previously been proposed for inhibiting and/or activating PKC as a whole, as methods of treatment; however, the Applicant has in this case identified the specific PKC isoforms responsible for the gap junction dysfunction in a neuroblastoma system and selectively targeted only these isoforms. This is meant to avoid much of the toxicity and side-effects of general PKC antagonists/agonists.

PKC isoforms may be specifically inhibited in different ways. As described in the Examples which follow, antibodies specific to a given isoform can be used. Alternatively, peptidomimetics designed as drugs can be used to specifically inhibit or activate PKC isoforms.

Most PKC inhibitors are unspecific in such that they are based on the competitive inhibition with ATP. ATP is necessary to the activity of all protein kinases including PKCs. Other inhibitors are based on competition with the regulatory effectors and address classes of PKCs not specific isoforms. It will be appreciated by those skilled in the art that many PKC inhibitors are known in the art, any of which may be used in the present invention.

It will also be appreciated that the invention will apply to other cell types where improper gating of the gap junction channels is due to phosphorylation of the connexin forming the channel by a PKC isoform to be determined(as in some breast cancers). For example, restoration of proper cell gating in some glioma cell lines requires only a single step.

Other applications include:
Limit of cell growth
Suppression of multidrug resistance which is known to be elicited by PKCalpha
Increase of the so-called bystander effect (in tumor gene therapies based on the Herpes thymidine kinase gene and genciclovir treatment, presently on trial)
Stroke and neuro-degeneration
Heart Arrhythmia
And numerous other diseases.

More specifically, the above mentioned effects of the first step occur as a result of activation of protein kinase A (PKA) by application of exogenous cAMP permeant analog.

Any compound that increases cAMP, an obligate effector of protein kinase A (PKA) will subsequently increase the PKA activity and among other effects, increase the expression and/or localization of connexins.

cAMP synthesis in cells depends upon the activity of an enzyme termed adenylate cyclase. cAMP synthesis is increased either via inhibition of phosphdiesterase enzymes or via activation of the adenylate cydase. Forskolin is an activator of adenylate cyclase among several others such as histamine, salbutanol, prostaglandin E2, PACAP (pituitary adenyl cyclase activator peptide).

Forskolin is extracted from the roots of a plant Coleus Forskolii. The procedure for the isolation of the compound was patented in India by S. V. Bhat (patent 142875, 1975). The disclosure of this patent is incorporated herein by reference.

Pharmaceutical use of forskolin encompasses treatments against asthma, acute heart failure, inflammation and other minor applications (ointments). It is sold under the name colforsin and its derivative colforsin daropate hydrochloride.

Salbutamol is used as a medication for asthma and veterinary medicine and prostaglandin E2 is used to induce labor in human. Since labor induction is highly dependent upon gap junction regulation.

Dosage for colforsin : extracts containing 18% forskolin, 50 mg 2-3 times daily.

Other substances can be substituted for forskolin and the other mentioned drugs in enhancing cAMP levels such as IBMX, isobutylmethylzanthine and CORETEC a phosphodiesterase inhibitor.

Dosage-for CORETEC, 2.5-5 μg per kg weight. Intravenously (as olprinone) single dose in the case of heart failure.

The second step of Cx43 expression and gap junction function/gating needs to be increased(not decreased)in order to limit cell proliferation. Such an increase is dependent upon inhibition of one or more of the selected PKC isoforms, alpha, beta I and beta II.

Alternatively, in one embodiment, the second step can be used to induce apoptosis (programmed cell death). For example, apoptosis can be induced by inhibiting p38 MAP kinase which is shown for the first time to be associated with restoration of gap junction function.

For the purpose of illustration only, this invention has mostly be described in relation, to neuroblastoma cells. However, it is understood by those skilled in the art, that the invention encompasses other systems as well. It is well known e.g from the literature reference to come that neuroblastoma cells behave in vitro like tumour cells behave in vivo. Acordingly, the neuroblastoma cell model is extrapolatable to other systems, and provides convincing evidence of operability in vivo. By inhibiting or activating one or several PKA and PKC isoforms simultaneously, as done for the neuroblastoma, gap junction intercellular communications will be restored. By doing so, it may contribute to the control of malignancies, arrhythmia, diabetes, neurodegenerative diseases, psoriasis and other diseases as therapy or combinational therapy. It will also be appreciated that different connexins will be involved in different tissues and will be associated with different diseases when defective.

Other references mentioned are Goekjian, P. et al., Current Medicinal Chemistry, 6:877-903 (1999); and Bowling, N. et al., Journal Of Molecular And Cellular Cardiology, 33(4):789-798 (2001).

All documents mentioned herein are incorporated by reference.

Assay for Testing Drug Candidates

It will be appreciated by those skilled in the art that the methodology for modulating gap junctions also serves as an assay for testing drug candidates, for treating diseases, disorders or conditions characterized by dysfunctional gap junction mediated intercellular communication, and/or which exhibit modulating properties towards the activity of protein kinase A and/or protein kinase C families as well as other protein kinases such as c/v-src and MAPKs.

In a first step, a drug candidate compound is administered to diseased cells having dysfunctional gap junction mediated intercellular communication and/or abnormal localization of connexin within the cells, for example, as determined by microscope imaging determination of localization of connexin in the cells' cytoplasm near to the nucleus(perinuclear location), and/or a low level of connexin expression. An observation of the increased transcription of connexin and a re-localization of the connexin to the cells's membrane, and/or a measurement of increased activity of a selected PKA isoform, as determined e.g. by immunostaining, followed by a quantitative analysis of the stain, denotes a positive result.

In a second step, a drug candidate compound is administered to diseased cells having dysfunctional gap junction mediated intercellular communication. An observation, of an improvement in gap junction mediated intercellular communication as measured e.g. by scrape loading dye transfer assay, and/or a measurement of inhibition or activation of one or more selected PKC isoforms, e.g. inhibition by Mabs, incorporation into cells by endocytosis, and anti-sense PKC alpha, denotes a positive result.

The two steps may be performed, individually, sequentially or simultaneously, depending upon the requirement. In some cases, only one step is required, while in others, both may be required. It may be enough to increase the level of expression of connexin, and/or localize the connexin to the cells' membrane e.g. by activation of a selected PKA isotype. In other cases, the gap junctions may be properly localized at the cells' membrane, but not properly gated to permit effective intercellular communication. In one situation, the gating with surrounding healthy tissue cannot be made. In another situation, the gating is made with surrounding tissue with which it is not supposed to communicate. In such cases, only the second type of drug candidate e.g. a selected modulator of one or more PKC isoforms (or other protein kinases), would be required.

### EXAMPLES

Prior to Treatment

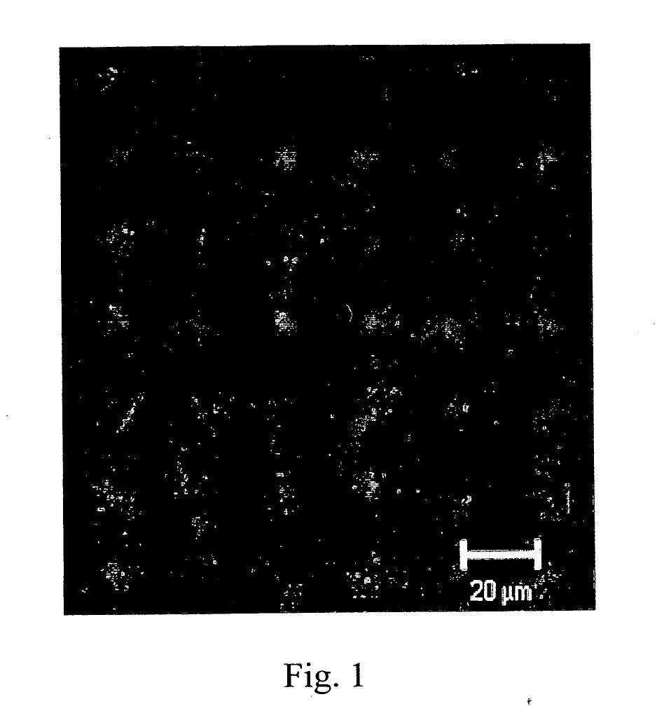

A confluent IMR32 cell culture was stained with a specific anti-connexin 43 antibody (Zymed) and visualized on a Zeiss confocal microscope. As seen in FIG. 1, the perinuclear localization of the connexin is clearly observed (the nucleus appears black). Very little connexin punctuation is observed at the cell boundaries while connexin staining around and above the nucleus and in the cytoplasm is abundant.

Treatment with a PKA Activator Only

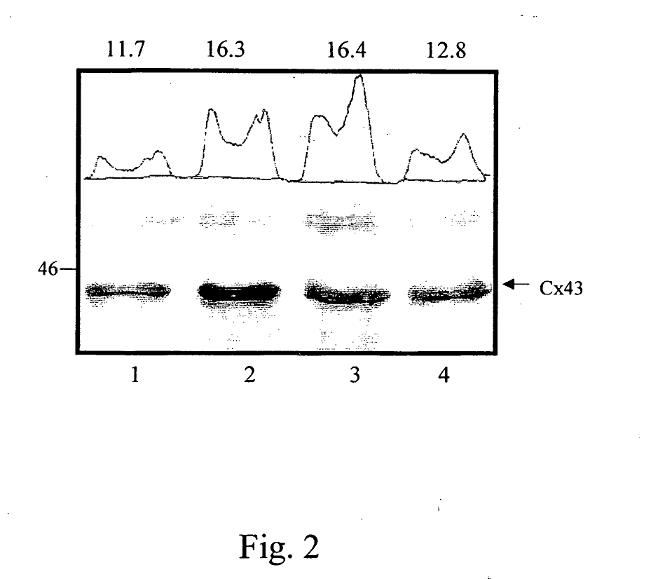

Referring to FIG. 2, whole cell extract was obtained from different cultures and 25 micro g of protein from each sample was separated by electrophoresis and electro-transferred onto nitrocellulase membranes. Detection was performed with an anti-connexin 43 selective antibody (Zymed). Cells treated with 8-bromo-cAMP (lane 2), an activator of PKA subtype I, show an increase in connexin 43 expression in comparison to control untreated cells (lane 1). The two unphosphorylated and phosphorylated connexin species increased unlike the ones expressed in cultures supplemented with all trans retinoic acid (RA) 1 and 10 micro M respectively (lanes 3 and 4). Similar results were obtained on two other neuroblastoma cell lines, namely SHSY5Y and SKNMC. Limited connexin 43 expression was also demonstrated on 14 biopsies obtained from the National Cancer Institute (USA).

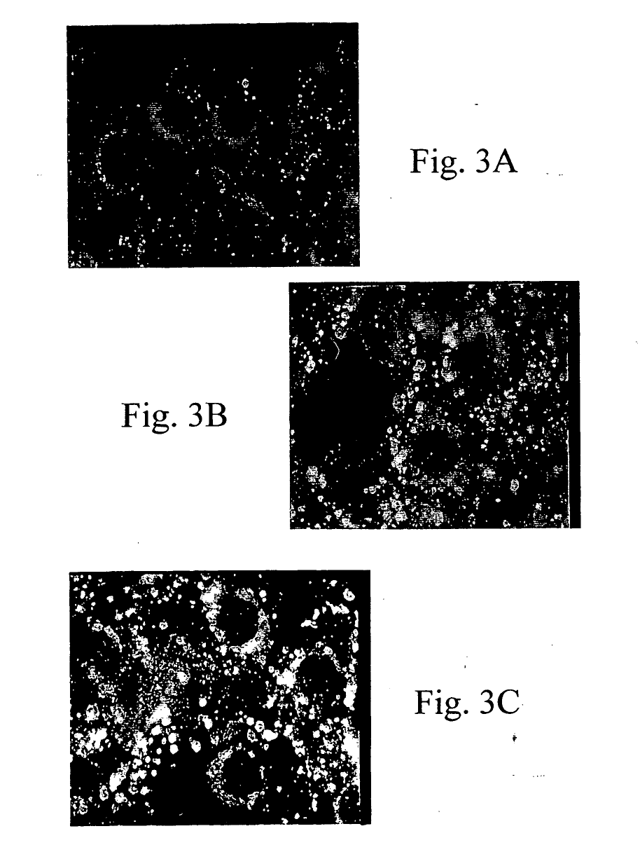

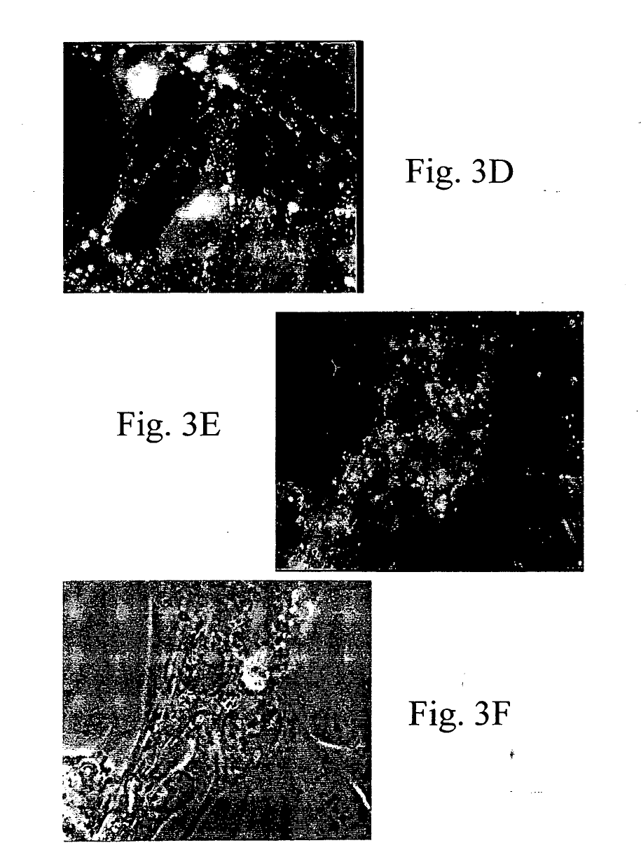

Referring to FIG. 3, SHSY5Y cell cultures (A), were supplemented with 8-bromo-cAMP (0.5 mM) for 1 day (B), 2 days (C), one week (D), and two weeks (E and F). Immunostaining was performed with a specific antibody to Cx43 and revealed with a secondary antibody conjugated to Alexa 488 (Molecular Probe). Progressively, the connexin is transferred from its perinuclear location to the cell border (B, C and D). After two weeks, the Cx43 still remains on the cell boundary (E) and is also located on the extended neurites (F). Original magnification was 40×. Similar results were obtained with IMR32 cells.

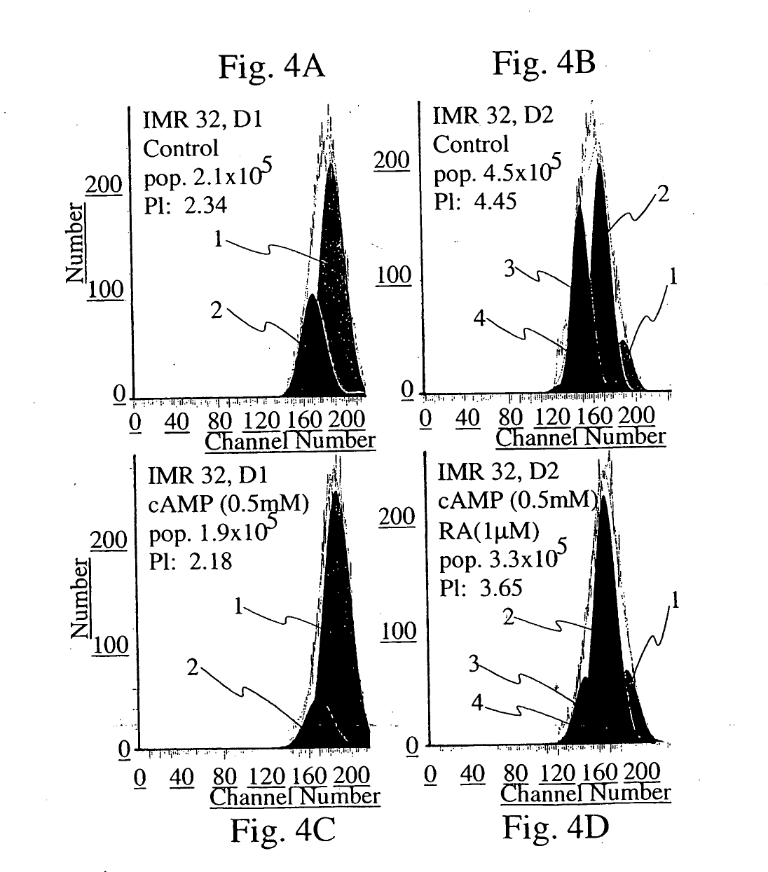

Referring to FIG. 4, confirmation of the negative regulatory effect on cell growth exerted by treatment of IMR32 cell cultures with 8-bromo-cAMP was obtained by FACS analysis of the populations and cell enumeration of untreated and treated cell cultures using the Trypan blue dye exclusion assay. An aliquot of each culture (10,000 cells) was analyzed by FACS on a Coulter Elite apparatus and ModFit software. The cells were labeled at time 0 with the membrane linker PKH26 (Sigma). The linker fluorescence decreases by half at each cell division. It allows for the quantitative estimation of the percentage of cells distributed as cell sub-populations having performed 2,3,4 . . . n divisions. The proliferation index (PI) is calculated from the data. In untreated controls, at day one, 2 cell sub-populations at division 2 and 3 (orange and green peak respectively) are present. At day 2 two, more peaks representing cells having divided 4 and 5 times emerge in those cells (red and blue respectively). In contrast, with exposure to 8bromo-cAMP, at day 1, the third division is delayed (reduced size of green peak). At day two, the delay in cell division increases. This is obvious from the tagging in orange peak reduction (division 2) and consecutive green peak increase (division 3). The division 4 red peak is considerably reduced in comparison to control and there are no cells at division 4 (blue peak). This is reflected in the size of the cell populations as a whole (pop) and the proliferative index (PI).

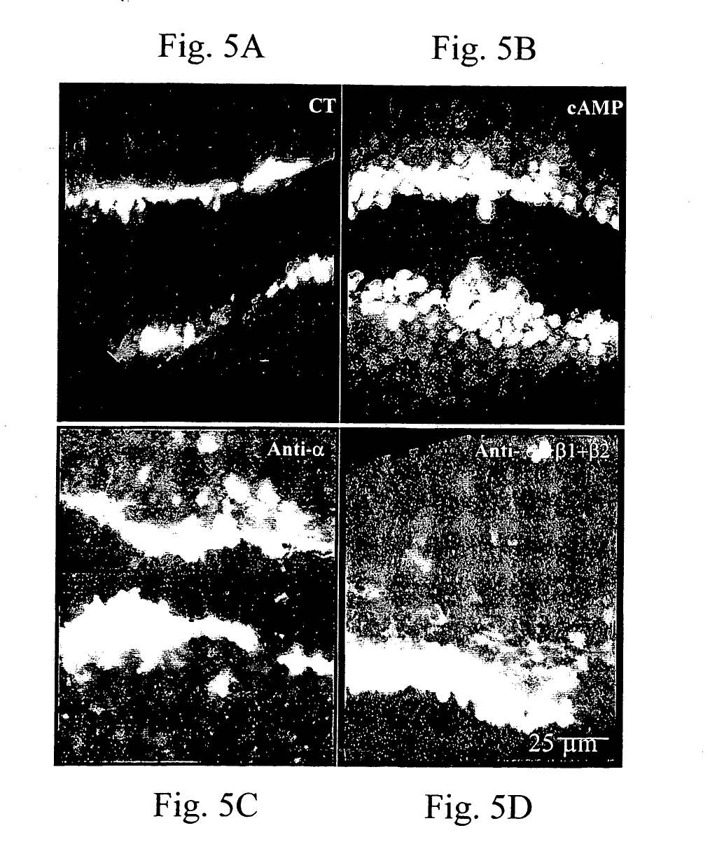

FIGS. 5A and 5B show that 8-bromo-cAMP treatment restores the normal sub-localization of connexin but does not restore full efficiency. Further, selective inhibition resulting from incorporation of antibodies to the PKC isoforms alpha and beta 1 and 2 inside the cells restores gap junction function. Cells carrying or not anti-PKC isoform specific antibodies were exposed for 48 h to 8-bromo-cAMP (except for the untreated controls) then assayed for gap junction function.

Treatment with Both a PKA Activator and One or More PKC Isoforms (Alpha, Beta I, and Beta II) Inhibitors

The classical scrape loading test was used to monitor the function of gap junction channels. Briefly, a cut is performed with a fine needle in sub-confluent cell cultures (refer reverse phases micrograph 5B). The culture is then covered with a film of fluorescent dye Lucifer yellow which is not cell permeant. The dye however penetrates the wounded cells along the scrape. From these loaded cells the dye can only be transferred to neighbor cells through the gap junction channels. The number of cell rows that receive the dye after a given time (3 min) provides an estimation of the gap junction function. Primary antibodies specific to PKC isoforms alpha beta I and beta II were introduced individually or in combination (as shown) by endocytosis using a kit from Molecular Probe following the supplier instructions. Controls for the introduction of the specific antibodies were performed by fixing and permeabilizing the cells then staining them with a secondary antibody conjugated to Texas red (Johnson labs). Only the incorporated primary antibodies will bind the secondary antibody conjugate (not shown but available).

CT illustrates the paucity of dye transfer in untreated proliferating cells. Cells treated with cAMP for two days exhibit more gap junction efficiency consistent with the effect on connexin expression and normal re-localization of the connexons (cAMP). Inhibiting selectively PKC alpha (Anti-alpha) greatly increases the dye transfer, note that the fluorescence lose intensity due to a larger diffusion of the dye. Combination incorporation of anti-alpha, −beta I and −beta II PKC isoforms exerts a potent positive effect on gap junction function (anti-alpha beta1+beta2). Reverse phase image confirms that all cultures were confluent (FIG. 5B).

The specific inhibition of other PKC isoforms like epsilon, Zeta and delta does not affect gap junction regulation (results not shown but available).

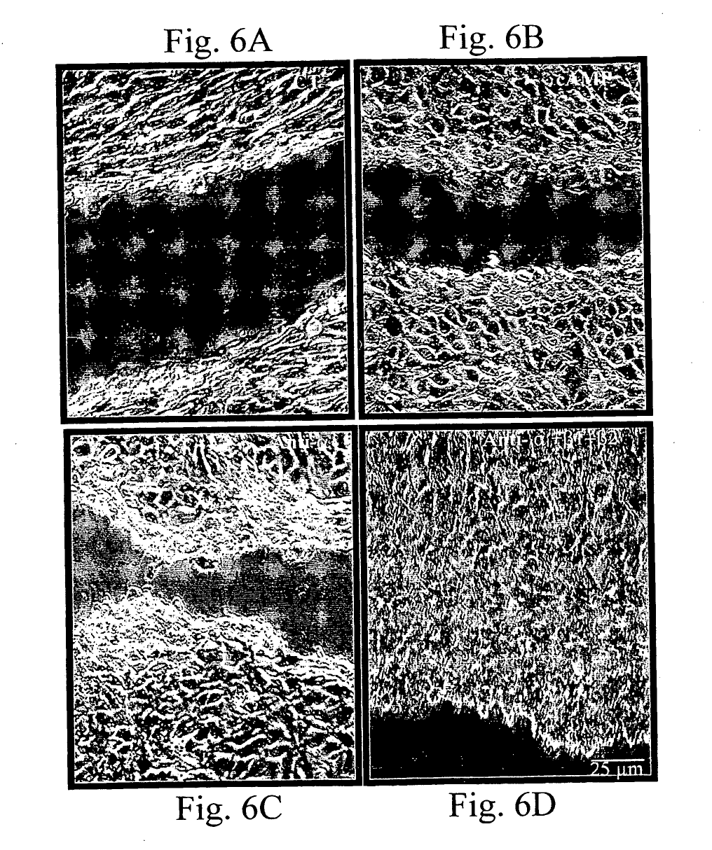

Referring to FIGS. 6A and 6B, the culture and experimental conditions are as in FIG. 5. Reverse phase showing that the cultures are confluent are shown as FIG. 6B. Neither Beta I, nor Beta II individual specific inhibition alter gap junction function estimated from dye transfer experiments (ant-betal; anti-beta II). However, combined specific inhibition of alpha PKC isoform with either anti-beta I or anti-beta II isoform slightly enhances dye transfer (antialpha+betaI, anti alpha+beta II) over anti-alpha alone. FIGS. 6A and 6B show that the combined inhibition of PKC alpha and beta is slightly more efficient in restoring gap junction function than individual inhibition of the isoforms beta I and betaII.

Treatment with Both a PKA Activator and a p38 MAP Kinase Inhibitor

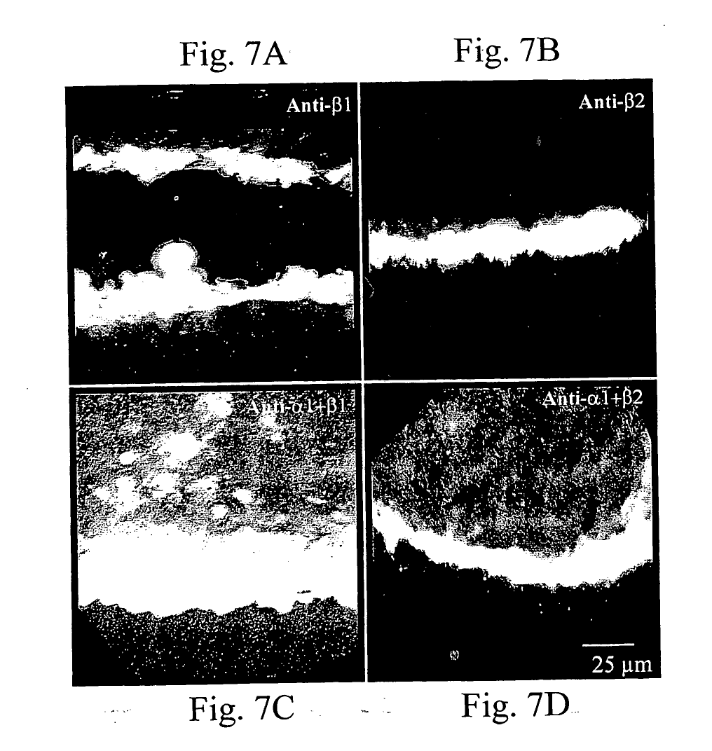

Referring to FIG. 7, the figure illustrates the positive effect on gap junction function of an inhibitor of the p38 MAP kinase, using the scrape loading assay. The experiment was performed on IMR32, similar results were obtained with the neuroblastoma cell line SHSY5Y. Control cell gap junction function following 8-bromo-cAMP exposure is shown in A. Gap junction function resuming following exposure of the culture to p38 inhibitor SB202190 (Chemicon) for 24 h is shown in B. The cells further died of apoptosis (results not shown but available). All documents referred to are incorporated herein by reference.

Treatment with a PKC Inhibitor

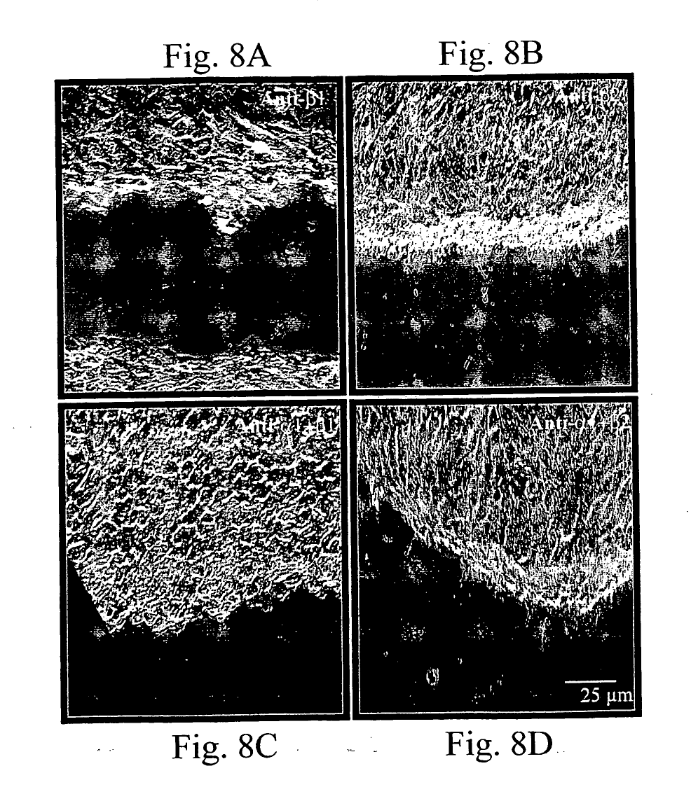

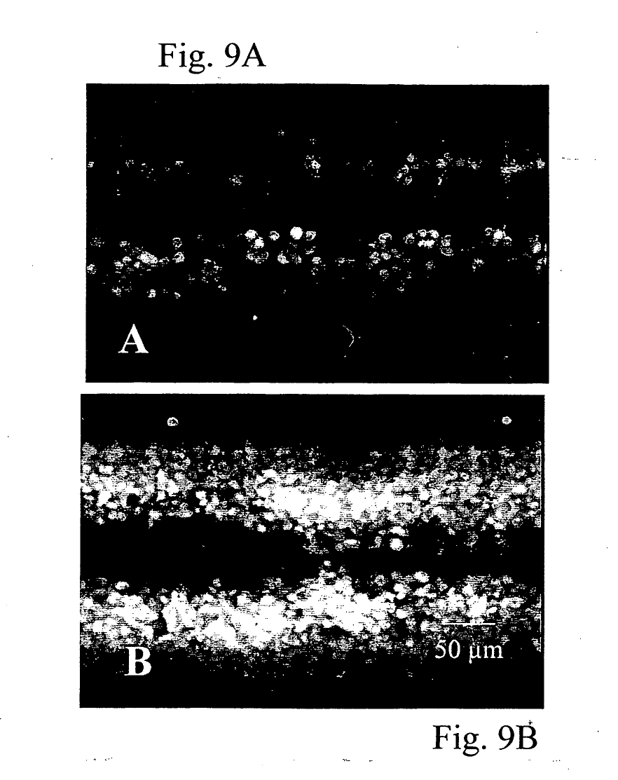

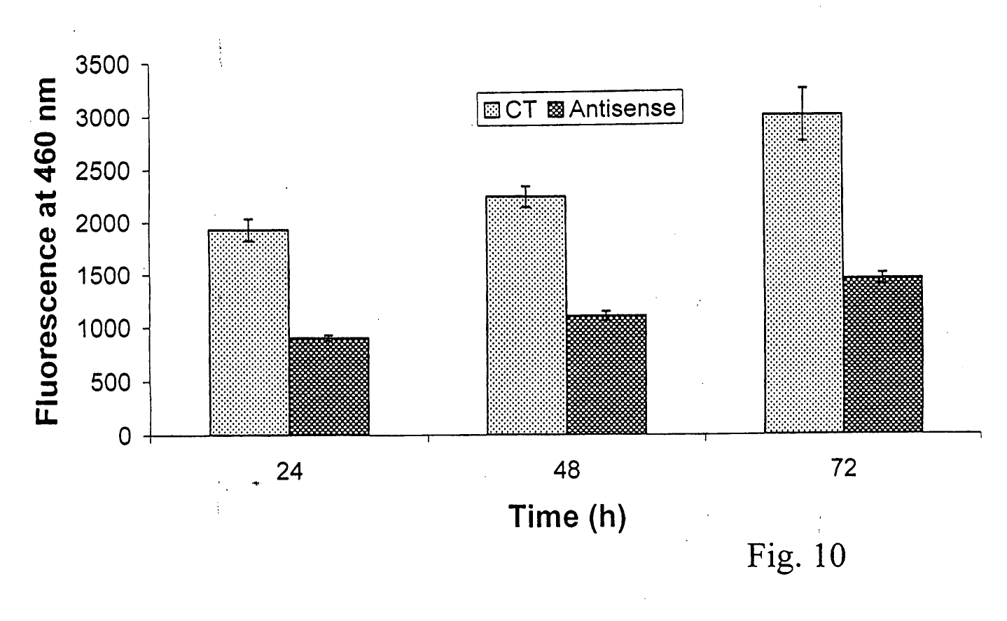

FIG. 8 shows the effect of PKC alpha RNA antisense on growth of IMR32 cells. In order to directly confirm that PKC alpha inhibition and associated gap junction channel tunction recovery resulted in the negative regulation of neuroblastoma cell growth, the cells received the specific RNA antisense towards the PKC alpha isoform (it prevents the formation of the targeted protein, here the PKC isoform alpha). Cell growth was measure as described in Tables 1, 2, and 3. The growth inhibition is even increased when compared to the results reported in Table 3. The kinetics are, however, similar.

It will be appreciated by those skilled in the art that although Mabs would not be used in vivo in the treatment of animals, nevertheless, this in vitro model using PKC alpha RNA anti-sense, provides convincing proof of concept for the use of novel very selective PKC inhibitors in vivo (others than the specific RNA antisense of the PKC isoforms). Two important therapeutic applications of such inhibitors are the bystander effect that relies on restoration of gap junction functions and multidrug resistance that relies on the inhibition of PKC alpha activity.

Although we have not specifically shown that we overcome drug resistance, it is a well known, published fact (by others) that it is controlled by the PKC alpha isoform. Similarly, it is well known that gap junction function is a prerequisite to the bystander effect,as explained above. Accordingly, it will be appreciated by those skilled in the art that that such effects are readily extrapolatable from the evidence provided herein.

TABLE 1
cAMP analog treatment negatively regulates the growth of IMR32
neuroblastoma cells in vitro: comparison with RA treatment.

Treatment

| Time/day | Control | A1 | A2 | A2 + RA | A1 + RA |
|---|---|---|---|---|---|
| 1 | 100 | 89 | 97 | 101 | 84 |
| 2 | 100 | 104 | 88 | 77 | 87 |
| 3 | 100 | 103 | 90 | 82 | 90 |
| 4 | 100 | 100 | 50 | 42 | 63 |
| 5 | 100 | 101 | 47 | 56 | 70 |
| 6 | 100 | 104 | 37 | 46 | 68 |

Legend: IMR32 neuroblastoma cells were seeded in 96 well plates at a density of 1000 cells per well. Treatments were as follows: Control cells are untreated and growing in a MEM-based proliferation medium. A1 cells were switched to the differentiation medium; A2 cells were grown in the A1 medium supplemented on day 1 with 8-bromo cAMP (a permeant cAMP analog) 0.5 mM. A2+RA treatment consisted of medium A2 supplemented with all trans retinoic acid (RA) 1 uM on day 2; A1+RA consist of RA supplementation 1 uM in the absence of the cAMP analog. The population size (24 replica per treatment) was determined by a procedure using Hoechst as a reagent and cytofluor (BioRad) fluorometer for measurement. The results are expressed as a percent of matching controls. It illustrates that addition of cAMP reduces growth (A2), that the effect of RA treatment on growth is not additive (A2+RA). RA treatment by itself is not very efficient in affecting growth (A1+RA).

TABLE 2
Kinetics of PCNA expression in IMR32 neuroblastoma cells
following activation of PKA by 8-bromo-cAMP. Comparison
with RA treatment.

| | Time/day | | | | | |
|---|---|---|---|---|---|---|
| | Day1 | | Day3 | | Day5 | |
| Treatment | % Pop | Mean I | % Pop | Mean I | % Pop | Mean I |
| A1 | 97.9 | 16.2 | 96.8 | 16.9 | 98.1 | 11.0 |
| A2 | 98.2 | 19.1 | 98.7 | 6.2 | 96.7 | 7.2 |
| A2 + RA | 98.9 | 19.7 | 99.0 | 8.0 | 98.7 | 9.6 |

Legend: PCNA is an effector of DNA polymerase and a commonly used marker of tumorigenicity. It is expressed in all phases of the t during active proliferation (A1) PCNA expression is relatively high in all samples. 3 days after treatment supplementation of the cultures with 8-bromo cAMP provided either individually or with RA decreased the PCNA expression, and consequently the % of cells in S phase by 2 third confirming the inhibitory effect observed in Table 1.

TABLE 3
Restoration of Gap Junction function and growth inhibition
of the cancer neuroblastoma cells are associated.

| | Day 1 | | | | Day 3 | | | | Day 4 | | | |
|---|---|---|---|---|---|---|---|---|---|---|---|---|
| | DNA | | | | DNA | | | | DNA | | | |
| | content | | | | content | | | | content | | | |
| | CT | T | % CT | % IN | CT | T | % CT | % IN | CT | T | % CT | % IN |
| Anti-α | 997 | 927 | 93 | 7 | 1439 | 1161 | 80.7 | 19.3 | 1918 | 1298 | 67.7 | 32.3 |
| Anti-βI,II | 770 | 750 | 97.4 | 2.6 | 1184 | 1081 | 91.3 | 8.7 | 1700 | 1512 | 88.9 | 11.1 |
| Anti-α + βI,II | 951 | 903 | 95 | 5 | 1358 | 995 | 73.3 | 16.7 | 1528 | 724 | 47.4 | 52.6 |

Legend: Populations growth is estimated as described in tables 1 and 2. Values are fluorescence intensities. The values are directly proportional to the total DNA content and number of cells. Controls were treated following endocytosis protocols (as described in FIG. 5 and 6) but did not receive primary antibodies. The treated samples incorporated anti PKC alpha, betaI, BetaII or a combination as indicated. CT=controls; T=anti-isoform treatment; % CT is the percentage of growth of treated samples versus that of matching controls; % IN is the percentage of growth inhibition exerted by the anti-isoform (s).

1. A method of modulating gap junction mediated intercellular communication in vertebrate cells characterized by dysfunctional gap junction communication, comprising a step of providing the cells with a compound that re-localizes the gap junctions at the cells' membrane.

2. A method according to claim 1, where the compound also increases the expression level of connexin in the cells.

3. A method according to claim 1 or wherein the compound is an activator of a selected protein kinase A (PKA) isoform.

4. A method according to claim 1, including the additional step of providing the cells with another compound that restores gap junction gating.

5. A method according to claim 4, wherein the another compound is a modulator for specifically modulating one or more selected PKC isoforms.

6. A method according to claim 4 wherein the step and the additional step are preformed sequentially or simultaneously.

7. A method of modulating gap junction mediated intercellular communication in vertebrate cells characterized by dysfunctional gap junction communication, comprising a step of providing the cells with a compound that restores gap junction gating.

8. A method according to claim 7, wherein the compound is a modulator for specifically modulating one or more selected PKC isoforms.

9. A method of modulating intercellular gap junction communication in an animal between diseased cell tissue and adjacent healthy cell tissue, comprising the steps of (a) providing the cells with a compound that re-localizes the gap junctions at the cells' membrane, and (b) providing the cells with another compound that restores intercellular gap junction gating.

10. A method according to claim 1, wherein the cells are cancer cells.

11. A method according to claim 10, wherein the cancer cells are neuroblastoma cells.

12. A method according to claim 11, wherein the PKA isoform that is activated is PKA sub-type I, PKA sub-type II or a mixture thereof.

13. A method according to claim 12, wherein the PKA activator is forskolin, sulbutamol, isobutylmethylzanthine or phosphodiesterase inhibitors.

14. A method according to claim 11, wherein the PKC modulator is a PKC inhibitor.

15. A method according to claim 14, wherein the PKC inhibitor is an inhibitor of one or more of PKC isoforms alpha, beta I and beta II.

16. A method according to claim 2, wherein the connexin is connexin 43.

17. A method of treating a cancer tumour in a patient, comprising the steps of:
(a) providing the cell with a protein kinase A (PKA) activator; and
(a) providing the cell with modulator for specifically inhibiting or activating one or more PKC isoforms.

18. An assay for testing drug candidates for treating diseases, disorders or conditions characterized by dysfunctional gap junction mediated intercellular communication, and/or which exhibit modulation of the activity of protein kinase A, comprising a step of administering a drug candidate compound to cells having dysfunctional gap junction mediated intercellular communication and/or abnormal localization within the cells of connexin, and/or a low level of connexin expression, wherein a quantitative estimation of an increase in the transcription of connexin e.g. by Western blot, and a re-localization of the connexin to the cells's membrane, and/or a measurement of increased activity of a selected PKA isoform, as determined e.g. by immunostaining, followed by microscope imaging analysis, denotes a positive result.

19. An assay for testing drug candidates for treating diseases, disorders or conditions characterized by dysfunctional gap junction mediated intercellular communication, and/or which exhibit modulation of the activity of protein kinase C, comprising a step of administering a drug candidate compound to cells having dysfunctional gap junction mediated intercellular communication, wherein an observation, of an improvement in gap junction mediated intercellular communication as measured e.g. by scrape loading dye transfer assay, and/or a measurement of inhibition or activation of one or more selected PKC isoforms, denotes a positive result.
